(19) United States
(12) Patent Application Publication
(10) Pub. No.: US 2008/0085259 A1
(43) Pub. Date: Apr. 10, 2008

(54) Amine condensation polymers as phosphate sequestrants

(76) Inventors: Chad C. Huval, Somerville, MA (US); Stephen Randall Holmes-Farley, Arlington, MA (US); Pradeep K. Dhal, Westford, MA (US)

Correspondence Address: GENZYME CORPORATION;LEGAL DEPARTMENT, 15 PLEASANT ST CONNECTOR, FRAMINGHAM, MA, 01701-9322, US

(21) Appl. No.: 11/799,739

(22) Filed: May 2, 2007

Related U.S. Application Data
(60) Provisional application No. 60/797,966, filed on May 5, 2006.

(51) Int. Cl. A61K 31/785; A61P 3/00; C08G 73/02

(52) U.S. Cl. 424/78.35; 525/540; 528/422

(57) ABSTRACT

Disclosed is a polymer or physiologically acceptable salt thereof. The polymer comprises a polymerized multifunctional amine monomer. The amine monomer comprises at least two amine groups and at least two acyclic nitrogen atoms that are connected through a —CH₂CH₂— group, provided that the amine monomer is not ethylenediamine or diethylenetriamine. The disclosed polymers can be used to bind anions in subject in need of such treatment.

### RELATED APPLICATIONS

This application claims the benefit of U.S. Provisional Application No. 60/797,966, filed on May 5, 2006, the entire teachings of which are incorporated herein by reference.

### BACKGROUND OF THE INVENTION

Hyperphosphatemia frequently accompanies diseases associated with inadequate renal function, hypoparathyroidism, and certain other medical conditions. Hyperphosphatemia is typically defined as possessing a serum phosphate level of over about 6 mg/dL. The condition, especially if present over extended periods of time, leads to severe abnormalities in calcium and phosphorus metabolism and can be manifested by aberrant calcification in joints, lungs, and eyes.

Therapeutic efforts to reduce serum phosphate include dialysis, reduction in dietary phosphate, and oral administration of insoluble phosphate binders to reduce gastrointestinal absorption. Dialysis and reduced dietary phosphate are generally unsuccessful in adequately reversing hyperphosphatemia. Further difficulties in these therapeutic regimens include the invasive nature of dialysis and the difficulties in modifying dietary habits in the latter therapy.

The oral administration of certain phosphate binders has also been suggested. Phosphate binders include calcium or aluminum salts. Calcium salts have been widely used to bind intestinal phosphate and prevent absorption. The ingested calcium CaHPO₄, or Ca(H₂PO₄)₂. Different types of calcium salts, including calcium carbonate, acetate (such as PhosLo® calcium acetate tablets), citrate, alginate, and ketoacid salts have been utilized for phosphate binding. This class of therapeutics generally results in hypercalcemia due to absorption of high amounts of ingested calcium. Hypercalcemia has been indicated in many serious side effects, such as cardiac arrhythmias, renal failure, and skin and visceral calcification. Frequent monitoring of serum calcium levels is required during therapy with calcium-based phosphate binders.

Aluminum-based phosphate binders, such as Amphojel® aluminum hydroxide gel, have also been used for treating hyperphosphatemia. These compounds complex with intestinal phosphate to form highly insoluble aluminum phosphate; the bound phosphate is unavailable for absorption by the patient. Prolonged use of aluminum gels leads to accumulations of aluminum, and often to aluminum toxicity, accompanied by such symptoms as encephalopathy, osteomalacia, and myopathy.

Selected ion exchange resins have also been suggested for use in binding phosphate. Those tested include Dowex® anion-exchange resins in the chloride form, such as XF 43311, XY 40013, XF 43254, XY 40011, and XY 40012. These resins have several drawbacks for treatment of hyperphosphatemia, including poor binding efficiency, necessitating use of high dosages for significant reduction of absorbed phosphate.

Certain anion exchange polymers, such as sevelamer hydrochloride (as disclosed in U.S. Pat. No. 5,667,775), have shown effectiveness as a phosphate sequestrant capable of lowering elevated serum phosphate levels. Sevelamer hydrochloride includes a polymer having pendent groups therefrom, the pendent groups having a single amino group.

It would be desirable to develop new polymers with similar or more favorable phosphate binding properties.

### SUMMARY OF THE INVENTION

Disclosed herein are novel polymers that bind anions, typically phosphate, and can therefore be used to remove target anions from a subject in need of such treatment.

One embodiment of the invention is a polymer or physiologically acceptable salt thereof which comprises a polymerized multifunctional amine monomer (hereinafter “amine monomer”). In one embodiment, the amine monomer comprises at least two amine groups and at least two acyclic nitrogen atoms that are connected through a —CH₂CH₂— group, provided that the amine monomer is not ethylenediamine or ethylenetriamine. In another embodiment, the amine monomer is represented by Structural Formula (I): 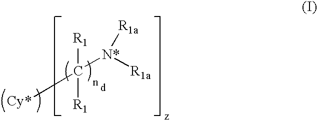
(Cy) is a C₄-C₁₀ saturated or unsaturated carbocyclic ring that is optionally substituted;
z is 2, 3 or 4.

Each R₁, independently, is H or an optionally substituted alkyl group or an optionally substituted aryl group, or forms together with an R₁ bonded to an adjacent carbon or nitrogen atom and their intervening atoms an optionally substituted alicyclic, aromatic, or heterocyclic group.

Each R₁ₐ, independently, is R₁,

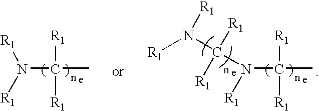

The nitrogen atom designated with “*” is optionally quarternized with R₁ₐ; and each n_{d}, independently, is 0 or is an integer from 1 to 10 and each nₑ is an integer from 2 to 10.

Another embodiment of the invention is a polymer or physiologically acceptable salt thereof which comprises an amine-containing repeat unit (referred to herein as an “amine repeat unit”). In one embodiment, the amine repeat unit comprises at least two amine groups and at least two acyclic nitrogen atoms that are connected through a —CH₂CH₂— group, provided that the repeat unit is not —NHCH₂CH₂NH—, —NHCH₂CH₂NHCH₂CH₂NH—, —NHCH₂CH₂(N—)CH₂CH₂NH—, or —NHCH₂CH₂(N—)CH₂CH₂NH₂.

In another embodiment, the amine repeat unit is represented by Structural Formula (II):

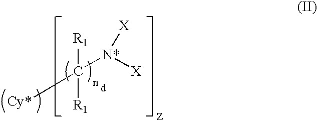

The polymer is crosslinked with multifunctional crosslinking groups.

(Cy) is a C₄-C₁₀ saturated or unsaturated carbocyclic ring that is optionally substituted.

z is 2, 3 or 4.

Each R₁, independently, is H or an optionally substituted alkyl group or an optionally substituted aryl group, or forms together with an R₁ bonded to an adjacent carbon or nitrogen atom and their intervening atoms an optionally substituted alicyclic, aromatic, or heterocyclic group.

Two or more of the groups represented by X are each a covalent bond to another atom in the polymer and the remainder of the groups represented by X are R₁.

The nitrogen atom designated with “*” is optionally quarternized with R₁ or

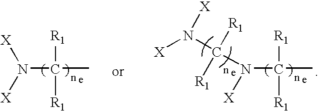

Each n_{d}, independently, is 0 or an integer from 1 to 10 and nₑ is an integer from 2 to 10.

Another embodiment of the present invention is a method for removing a target anion from a subject. The method comprises administering an effective amount of a polymer disclosed herein or physiologically acceptable salt thereof to the subject.

Another embodiment of the invention is directed to a pharmaceutical composition comprising a pharmaceutically acceptable carrier or diluent; and a polymer disclosed herein or a pharmaceutically acceptable salt thereof. The pharmaceutical composition is used for medicinal therapy.

Another embodiment of the invention is the use of a disclosed polymer or a physiologically acceptable salt thereof for the manufacture of a medicament for removing a target anion from a subject.

Yet another embodiment of the invention is a method for controlling serum phosphate in a patient suffering from hyperphosphatemia comprising administering to the patient a pharmaceutical composition comprising a polymer disclosed herein or a physiologically acceptable salt thereof and a pharmaceutically acceptable carrier or diluent.

### DETAILED DESCRIPTION OF THE INVENTION

The invention is directed to a polymer or physiologically acceptable salt thereof which comprises a polymerized amine monomer. The amine monomer comprises at least two amine groups and at least two acyclic nitrogen atoms that are connected through a —CH₂CH₂— group, provided that the amine monomer is not ethylenediamine or ethylenetriamine. In more specific embodiments, the amine monomer comprises at least three nitrogen atoms and more typically at least four nitrogen atoms.

In a specific embodiment, the amine monomer is represented by Structural Formula (III).

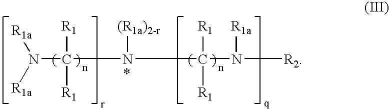

Values and preferred values for the variables in Structural Formula (III) are defined in the following six paragraphs.

Each R₁, independently, is H or an optionally substituted alkyl group or an optionally substituted aryl group, or forms together with an R₁ bonded to an adjacent carbon or nitrogen atom and their intervening atoms an optionally substituted alicyclic, aromatic, or heterocyclic group. Preferably, each R₁, independently, is H or an optionally substituted alkyl group or an optionally substituted aryl group. More preferably, each R₁, independently, is H or an alkyl group optionally substituted with —OH, alkoxy, halogen, or a phenyl or pyridyl group, wherein the phenyl and pyridyl groups are optionally substituted with —OH, alkoxy, halogen, haloalkyl or haloalkoxy.

Each R^{1a} is independently R₁ or 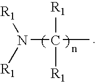
Preferably, each R₁ₐ is R₁.

R₂ is R^{1a} or a group represented by the following structural formula: 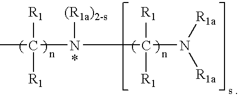
In a more specific embodiment, each R₂ is R₁ₐ. Alternatively, each R₂, independently, is H or an alkyl group optionally substituted with —OH, alkoxy, halogen or a phenyl group optionally substituted with —OH, alkoxy, halogen, haloalkyl, haloalkoxy.

Each nitrogen atom designated with “*” is optionally quarternized with R₁ₐ.

q is 0 or an integer from 1 to 10; r and s are 0, 1, or 2 with the proviso that the sum of r, s and q is greater than 1.

Each n, independently, is an integer from 2 to 10 with the proviso that at least one n is 2. Preferably, n is 2.

In a more specific embodiment, the amine monomer is represented by a structural formula selected from Structural Formulas (IV)-(VI):

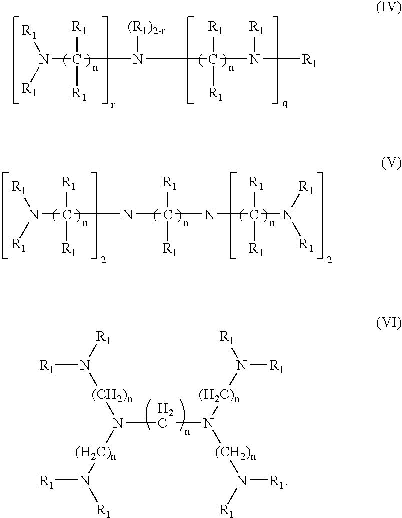

The variables in Structural Formulas (IV)-(VI) are as defined in Structural Formula (III).

In another specific embodiment, the amine monomer is represented by Structural Formula (VII): 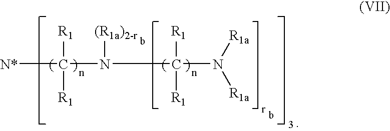
The variables for Structural Formula (VII) are defined in the following 5 paragraphs.

Each R₁, independently, is H or an optionally substituted alkyl group or an optionally substituted aryl group, or forms together with an R₁ bonded to an adjacent carbon or nitrogen atom and their intervening atoms an optionally substituted alicyclic, aromatic, or heterocyclic group. Preferably, each R₁, independently, is H or an optionally substituted alkyl group or an optionally substituted aryl group. More preferably, each R₁, independently, is H or an alkyl group optionally substituted with —OH, alkoxy, halogen, or a phenyl or pyridyl group, wherein the phenyl and pyridyl groups are optionally substituted with —OH, alkoxy, halogen, haloalkyl or haloalkoxy.

Each R^{1a} is independently R₁ or 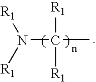
Preferably, each R₁ₐ is R¹.

Each nitrogen atom designated with “*” is optionally quarternized with R^{1a}.

Each r_{b}, independently, is 0, 1, or 2.

Each n, independently, is an integer from 2 to 10 with the proviso that at least one n is 2. Preferably, n is 2.

In a more specific embodiment, the amine monomer is represented by Structural Formulas (VIII): 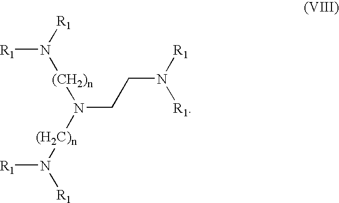
The variables in Structural Formula (VIII) are as described in Structural Formula (VII).

In another specific embodiment, the amine monomer is represented by Structural Formula (IX): 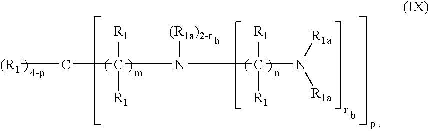
The variables for Structural Formula (IX) are defined in the following five paragraphs.

Each R₁, independently, is H or an optionally substituted alkyl group or an optionally substituted aryl group, or forms together with an R₁ bonded to an adjacent carbon or nitrogen atom and their intervening atoms an optionally substituted alicyclic, aromatic, or heterocyclic group. Preferably, each R₁, independently, is H or an optionally substituted alkyl group or an optionally substituted aryl group. More preferably, each R₁, independently, is H or an alkyl group optionally substituted with —OH, alkoxy, halogen, or a phenyl or pyridyl group, wherein the phenyl and pyridyl groups are optionally substituted with —OH, alkoxy, halogen, haloalkyl or haloalkoxy.

Each R₁ₐ is independently R₁ or 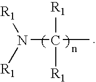
Preferably, each R₁ₐ is R₁.

p is 1, 2, 3, or 4; each r_{b}, independently, is 0, 1, or 2 with the proviso that r_{b} is 1 or 2 if p is equal to 1.

Each m, independently, is 0 or an integer from 1 to 10; and

each n, independently, is an integer from 2 to 10 with the proviso that at least one n is 2. Preferably, n is 2.

In a more specific embodiment, the amine monomer is represented by Structural Formula (X): 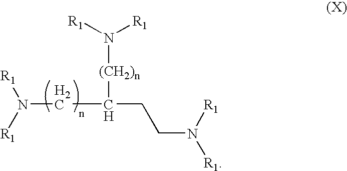
The variables in Structural Formula (X) are as described for Structural Formula (IX).

In another specific embodiment, the amine monomer is represented by Structural Formula (XI): 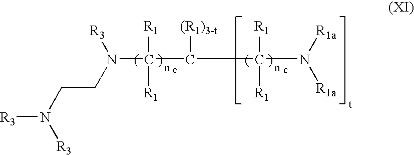
The variables in Structural Formula (IX) are described in the following six paragraphs.

Each R₁, independently, is H or an optionally substituted alkyl group or an optionally substituted aryl group, or forms together with an R₁ bonded to an adjacent carbon or nitrogen atom and their intervening atoms an optionally substituted alicyclic, aromatic, or heterocyclic group. Preferably, each R₁, independently, is H or an optionally substituted alkyl group or an optionally substituted aryl group. More preferably, each R₁, independently, is H or an alkyl group optionally substituted with —OH, alkoxy, halogen, or a phenyl or pyridyl group, wherein the phenyl and pyridyl groups are optionally substituted with —OH, alkoxy, halogen, haloalkyl or haloalkoxy.

Each R^{1a} is independently R₁ or

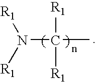

Each R₃, independently, is H, 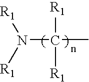
or an optionally substituted alkyl group or an optionally substituted aryl group. Preferably, each R₃, independently, is H or an alkyl group optionally substituted with —OH, alkoxy, or a phenyl or pyridyl group, wherein the phenyl and pyridyl groups are optionally substituted with —OH, alkoxy, halogen, haloalkyl or haloalkoxy.

Each t, independently, is 0, 1, 2, or 3.

Each n is an integer from 2 to 10. Preferably, n is 2.

Each n_{c}, independently, is 0 or an integer from 1 to 10.

In a more specific embodiment, the amine monomer is represented by Structural Formula (XII): 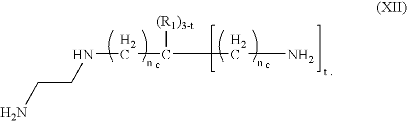
The variables in Structural Formula (XII) are as described for Structural Formula (XI).

Specific examples of suitable amine monomers include tris(2-aminoethyl)amine, triethylenetetramine, tetraethylenepentamine, pentaethylenehexamine, N-boc-ethylenediamine, tris[(methylamino)ethyl]amine, N,N,N′,N′-tetrakis(3-aminopropyl) 1,2-diaminoethane.

Another embodiment of the invention is a polymer or physiologically acceptable salt thereof comprising a polymerized amine monomer represented by Structural Formula (I): 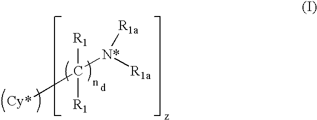
Values and preferred values for the variables in Structural Formula (I) are provided in the following six paragraphs.

(Cy) is a C₄-C₁₀ saturated or unsaturated carbocyclic ring. Preferably, (Cy) is a cyclohexyl optionally substituted with C₁-C₂ alkyl, hydroxyl, halogen or C₁-C₂ alkoxy or phenyl optionally substituted with —OH, alkyl, alkoxy, halogen, haloalkyl or haloalkoxy.

z is 2, 3 or 4. Preferably, z is 3 or 4.

Each R₁, independently, is H or an optionally substituted alkyl group or an optionally substituted aryl group, or forms together with an R₁ bonded to an adjacent carbon or nitrogen atom and their intervening atoms an optionally substituted alicyclic, aromatic, or heterocyclic group. Preferably, each R₁, independently, is H or an optionally substituted alkyl group or an optionally substituted aryl group. More preferably, each R₁, independently, is H or an alkyl group optionally substituted with —OH, alkoxy, halogen, or a phenyl or pyridyl group, wherein the phenyl and pyridyl groups are optionally substituted with —OH, alkoxy, halogen, haloalkyl or haloalkoxy.

Each R₁ₐ, independently, is R₁, 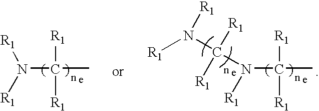
Preferably, each R₁ₐ is R₁.

The nitrogen atom designated with “*” is optionally quarternized with R₁ₐ.

Each n_{d}, independently, is 0 or an integer from 1 to 10. Preferably, each n_{d}, independently, is an integer from 1 to 10. Each nₑ is an integer from 2 to 10.

The invention is also directed to a polymer or physiologically acceptable salt thereof which comprises an amine repeat unit. The amine repeat unit comprises at least two amine groups and at least two acyclic nitrogen atoms that are connected through a —CH₂CH₂— group, provided that the repeat unit is not —NHCH₂CH₂NH—, —NHCH₂CH₂NHCH₂CH₂NH—, —NHCH₂CH₂(N—)CH₂CH₂NH—, or —NHCH₂CH₂(N—)CH₂CH₂NH₂. In more specific embodiments, the repeat unit comprises at least three nitrogen atoms and more typically at least four nitrogen atoms.

In a more specific embodiment, the amine repeat unit is represented by Structural Formula (XIII): 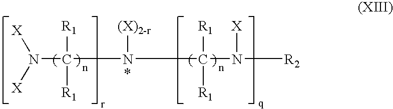
Values and preferred values for the variables in Structural Formula (XIII) are provided in the following six paragraphs.

Each R₁, independently, is H or an optionally substituted alkyl group or an optionally substituted aryl group, or forms together with an R₁ bonded to an adjacent carbon or nitrogen atom and their intervening atoms an optionally substituted alicyclic, aromatic, or heterocyclic group. Preferably, each R₁, independently, is H or an optionally substituted alkyl group or an optionally substituted aryl group. More preferably, each R₁, independently, is H or an alkyl group optionally substituted with —OH, alkoxy, halogen, or a phenyl or pyridyl group, wherein the phenyl and pyridyl groups are optionally substituted with —OH, alkoxy, halogen, haloalkyl or haloalkoxy.

Two or more of the groups represented by X are each a covalent bond to another atom in the polymer, and the remainder of the groups represented by X are R₁.

R₂ is X or a group represented by the following structural formula: 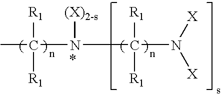
Preferably, each R₂, independently, is H or an optionally substituted alkyl group or an optionally substituted aryl group. More preferably, each R₂, independently, is H or an alkyl group optionally substituted with —OH, alkoxy, halogen, or a phenyl or pyridyl group, wherein the phenyl and pyridyl groups are optionally substituted with —OH, alkoxy, halogen, haloalkyl or haloalkoxy.

Each nitrogen atom designated with “*” is optionally quarternized with R₁ or

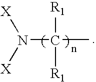

q is 0 or an integer from 1 to 10; r and s are 0, 1, or 2 with the proviso that the sum of r, s and q is greater than 1.

Each n, independently, is an integer from 2 to 10 with the proviso that at least one n is 2. Preferably, n is 2.

In a more specific embodiment, the amine repeat unit is represented by a structural formula selected from Structural Formulas (XIV)-(XXVI): 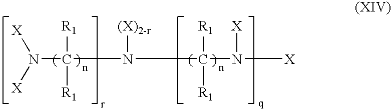 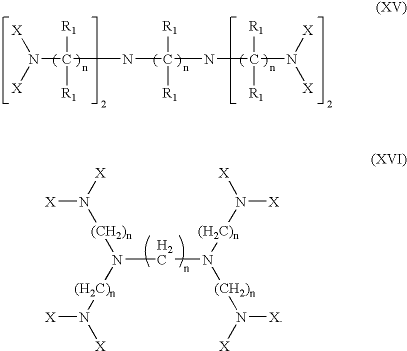
Values and preferred values for the variables in Structural Formulas (XIV)-(XVI) are as provided for Structural Formula (XIII).

In another specific embodiment, the amine repeat unit is represented by Structural Formula (XVII): 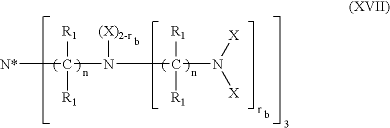
Values and preferred values for the variables in Structural Formula (XVII) are provided in the following five paragraphs.

Each R₁, independently, is H or an optionally substituted alkyl group or an optionally substituted aryl group, or forms together with an R₁ bonded to an adjacent carbon or nitrogen atom and their intervening atoms an optionally substituted alicyclic, aromatic, or heterocyclic group. Preferably, each R₁, independently, is H or an optionally substituted alkyl group or an optionally substituted aryl group. More preferably, each R₁, independently, is H or an alkyl group optionally substituted with —OH, alkoxy, halogen, or a phenyl or pyridyl group, wherein the phenyl and pyridyl groups are optionally substituted with —OH, alkoxy, halogen, haloalkyl or haloalkoxy.

Two or more of the groups represented by X are each a covalent bond to another atom in the polymer, and the remainder of the groups represented by X are R₁.

Each r_{b}, independently, is 0, 1, or 2.

Each nitrogen atom designated with “*” is optionally quarternized with R₁ or

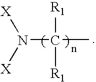

Each n, independently, is an integer from 2 to 10 with the proviso that at least one n is 2. Preferably, n is 2.

In a more specific embodiment, the amine repeat unit is represented by Structural Formulas (XVIII): 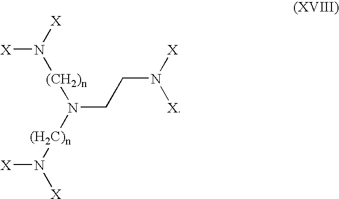
Values and preferred values for the variables in Structural Formula XVIII) are as provided for Structural Formula (XVII).

In another specific embodiment, the amine repeat unit is represented by Structural Formula (XIX): 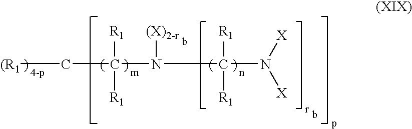
Values and preferred values for the variables in Structural Formula (XIX) are provided in the following five paragraphs.

Each R₁, independently, is H or an optionally substituted alkyl group or an optionally substituted aryl group, or forms together with an R₁ bonded to an adjacent carbon or nitrogen atom and their intervening atoms an optionally substituted alicyclic, aromatic, or heterocyclic group. Preferably, each R₁, independently, is H or an optionally substituted alkyl group or an optionally substituted aryl group. More preferably, each R₁, independently, is H or an alkyl group optionally substituted with —OH, alkoxy, halogen, or a phenyl or pyridyl group, wherein the phenyl and pyridyl groups are optionally substituted with —OH, alkoxy, halogen, haloalkyl or haloalkoxy.

Two or more of the groups represented by X are each a covalent bond to another atom in the polymer, and the remainder of the groups represented by X are R₁.

p is 1, 2, 3, or 4; each r_{b}, independently, is 0, 1, or 2 with the proviso that r_{b} is 1 or 2 if p is equal to 1.

Each m, independently, is 0 or an integer from 1 to 10; and

Each n, independently, is an integer from 2 to 10 with the proviso that at least one n is 2. Preferably, n is 2.

In a more specific embodiment, the amine repeat unit is represented by Structural Formula (XX): 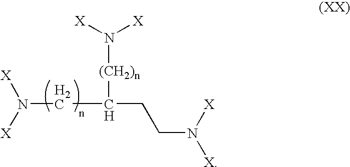
Values and preferred values for the variables in Structural Formula (XX) are as provided for Structural Formula (XIX).

In another specific embodiment, the amine repeat unit is represented by Structural Formula (XXI): 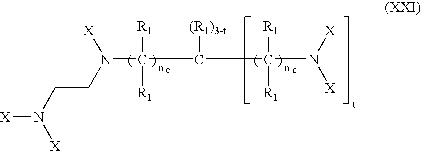
Values and preferred values for the variables in Structural Formula (XXI) are provided in the following four paragraphs.

Each R₁, independently, is H or an optionally substituted alkyl group or an optionally substituted aryl group, or forms together with an R₁ bonded to an adjacent carbon or nitrogen atom and their intervening atoms an optionally substituted alicyclic, aromatic, or heterocyclic group. Preferably, each R₁, independently, is H or an optionally substituted alkyl group or an optionally substituted aryl group. More preferably, each R₁, independently, is H or an alkyl group optionally substituted with —OH, alkoxy, halogen, or a phenyl or pyridyl group, wherein the phenyl and pyridyl groups are optionally substituted with —OH, alkoxy, halogen, haloalkyl or haloalkoxy.

Two or more of the groups represented by X are each a covalent bond to another atom in the polymer, and the remainder of the groups represented by X are R₁.

Each t, independently, is 0, 1, 2, or 3; and

Each n_{c}, independently, is 0 or an integer from 1 to 10.

In a more specific embodiment, the amine repeat unit is represented by Structural Formulas (XXII):

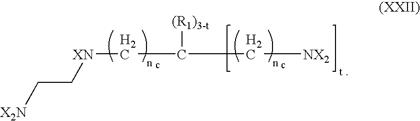

Values and preferred values for the variables in Structural Formula (XXII) are as provided for Structural Formula (XXI).

In another embodiment of the invention, the polymer of the invention comprises an amine repeat unit represented by Structural Formula (II) 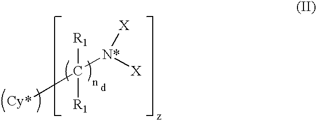
Values and preferred values for the polymerized monomer represented by Structural Formula (II) are provided in the following six paragraphs.

(Cy) is a C₄-C₁₀ saturated or unsaturated carbocyclic ring. Preferably, (Cy) is a cyclohexyl optionally substituted with C₁-C₂ alkyl, hydroxyl, halogen or C₁-C₂ alkoxy or phenyl optionally substituted with —OH, alkyl, alkoxy, halogen, haloalkyl or haloalkoxy.

z is 2, 3 or 4. Preferably, z is 3 or 4.

Each R₁, independently, is H or an optionally substituted alkyl group or an optionally substituted aryl group, or forms together with an R₁ bonded to an adjacent carbon or nitrogen atom and their intervening atoms an optionally substituted alicyclic, aromatic, or heterocyclic group.

Preferably, each R₁, independently, is H or an optionally substituted alkyl group or an optionally substituted aryl group. More preferably, each R₁, independently, is H or an alkyl group optionally substituted with —OH, alkoxy, halogen, or a phenyl or pyridyl group, wherein the phenyl and pyridyl groups are optionally substituted with —OH, alkoxy, halogen, haloalkyl or haloalkoxy.

Two or more of the groups represented by X are each a covalent bond to another atom in the polymer and the remainder of the groups represented by X groups are R₁. The nitrogen atom designated with “*” is optionally quarternized with R₁,

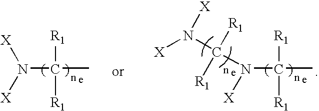

Each n_{d}, independently, is 0 or an integer from 1 to 10. Preferably, each n_{d}, independently, is an integer from 1 to 10. Each nₑ is an integer from 2 to 10.

A “multifunctional amine monomer” is a compound that comprises two or more amine groups and that can be reacted alone or with other compounds such that it is incorporated as a repeat unit into a polymer. A “polymerized multifunctional amine monomer” is a multifunctional amine monomer that has been reacted alone or with other compounds such that it has been incorporated into a polymer as a repeat unit. It is to be understood that when referring herein to a “polymerized multifunctional amine monomer”, the polymerized multi functional amine monomer is incorporated into the polymer by any suitable method, including, but not limited to, a single “polymerization” reaction, the stepwise addition of individual monomers via a series of reactions, the stepwise addition of blocks of monomers, or any combination of the foregoing. As noted above, the terms “multifunctional amine monomer” and “amine monomer” are used interchangeably herein.

The term “repeat unit” means a group in a polymer that repeats or appears multiple times in the polymer. An “amine repeat unit” is a repeat unit comprising one or more amine groups, preferably two or more amine groups.

The disclosed polymers include homopolymers which comprise no more than one type of polymerized monomer (or one type of repeat unit). Alternatively, the disclosed polymers include copolymers which comprise two different types of polymerized monomers (or two different types of repeat units). One or both of the polymerized monomers are polymerized amine monomers (or one or both of the repeat units are amine repeat units). Preferably, both of the polymerized amine monomers (or both of the amine repeat units) are described herein. In yet another alternative, the disclosed polymer comprises three or more different types of polymerized monomers (or three or more different types of repeat units).

The disclosed polymers are typically crosslinked with multifunctional crosslinking groups. The term “multifunctional crosslinking group” means a group which connects two or more repeat units or polymerized monomers within the polymer. Multifunctional crosslinking groups in the disclosed polymers are typically covalently bonded to the nitrogen atoms in the polymerized amine monomers or amine repeat units. In one option, the disclosed polymer comprises only one type of crosslinking group. Alternatively, the disclosed polymer comprises two or more different crosslinking groups.

The ratio of polymerized amine monomer to polymerized crosslinker in the disclosed polymer is typically from about 1:1 to about 1:6. For example, the ratio can be from about 1:1 to about 1:2, from about 1:1 to about 1:3, from about 1:1 to about 1:4, from about 1:1 to about 1:5, from about 1:2 to about 1:3, from about 1:2 to about 1:4, from about 1:2 to about 1:5, from about 1:2 to about 1:6, from about 1:3 to about 1:4, from about 1:3 to about 1:5, from about 1:3 to about 1:6, from about 1:4 to about 1:5, from about 1:4 to about 1:6 or from about 1:5 to about 1:6.

Multifunctional crosslinking groups in the disclosed polymers are typically formed from multifunctional crosslinking agents, which comprise two or more electrophilic groups capable of reacting and forming a covalent bond with a nitrogen atom. Examples of suitable electrophilic groups include halide, epoxide, acrylate, arylsulfonate and alkylsulfonate. Reaction of a multifunctional crosslinking agent with an amine monomer disclosed herein can form a disclosed polymer. The portion of a multifunctional crosslinking agent remaining after it reacts with the amine monomer forms a crosslinking group and is also referred to as the “residue of the crosslinking agent”. For example, —(CH₂)₆— is the crosslinking group formed from the crosslinking agent 1,6-dibromohexane and is also the residue of 1,6-dibromohexane.

Examples of suitable types crosslinking agents include dihaloalkane, haloalkyloxirane, alkyloxirane sulfonate, di(haloalkyl)amine, tri(haloalkyl)amine, diepoxide, triepoxide, tetraepoxide, bis(halomethyl)benzene, tri(halomethyl)benzene) and tetra(halomethyl)benzene.

Specific examples of crosslinking agents include epichlorohydrin, epibromohydrin, (iodomethyl)oxirane, glycidyl tosylate, glycidyl 3-nitrobenzenesulfonate, 4-tosyloxy-1,2-epoxybutane, bromo-1,2-epoxybutane, 1,2-dibromoethane, 1-bromo-2-chloroethane, 1,3-dibromopropane, bis(2-chloroethyl)amine, tris(2-chloroethyl)amine, and bis(2-chloroethyl)methylamine, 1,3-butadiene diepoxide, 1,5-hexadiene diepoxide, diglycidyl ether, 1,2,7,8-diepoxyoctane, 1,2,9,10-diepoxydecane, ethylene glycol diglycidyl ether, propylene glycol diglycidyl ether, 1,4-butanediol diglycidyl ether, glycerol diglycidyl ether, 1,3-diglycidyl glyceryl ether, N,N-diglycidylaniline, neopentyl glycol diglycidyl ether, diethylene glycol diglycidyl ether, 1,4-bis(glycidyloxy)benzene, resorcinol digylcidyl ether, 1,6-hexanediol diglycidyl ether, trimethylolpropane diglycidyl ether, 1,4-cyclohexanedimethanol diglycidyl ether, 1,3-bis-(2,3-epoxypropyloxy)-2-(2,3-dihydroxypropyloxy)propane, 1,2-cyclohexanedicarboxylic acid diglycidyl ester, 2,2′-bis(glycidyloxy)diphenylmethane, bisphenol F diglycidyl ether, 1,4-bis(2′,3′-epoxypropyl)perfluoro-n-butane, 2,6-di(oxiran-2-ylmethyl)-1,2,3,5,6,7-hexahydropyrrolo[3,4-f]isoindol-1,3,5,7-tetraone, bisphenol A diglycidyl ether, ethyl 5-hydroxy-6,8-di(oxiran-2-ylmethyl)-4-oxo-4h-chromene-2-carboxylate, bis[4-(2,3-epoxy-propylthio)phenyl]-sulfide, 1,3-bis(3-glycidoxypropyl)tetramethyldisiloxane, 9,9-bis[4-(glycidyloxy)phenyl]fluorene, triepoxyisocyanurate, glycerol triglycidyl ether, N,N-diglycidyl-4-glycidyloxyaniline, isocyanuric acid (S,S,S)-triglycidyl ester, isocyanuric acid (R,R,R)-triglycidyl ester, triglycidyl isocyanurate, trimethylolpropane triglycidyl ether, glycerol propoxylate triglycidyl ether, triphenylolmethane triglycidyl ether, 3,7,14-tris[[3-(epoxypropoxy)propyl]dimethylsilyloxy]-1,3,5,7,9,11,14-heptacyclopentyltricyclo[7,3,3,15,11]heptasiloxane, 4,4′-methylenebis(N,N-diglycidylaniline), bis(halomethyl)benzene, bis(halomethyl)biphenyl and bis(halomethyl)naphthalene.

The disclosed polymers include those comprising polymerized tris(2-aminoethyl)amine, triethylenetetramine, tetraethylenepentamine, pentaethylenehexamine, N-boc-ethylenediamine, tris[(methylamino)ethyl]amine and N,N,N′,N′-tetrakis(3-aminopropyl)1,2-diaminoethane crosslinked with epichlorohydrin, 1,2-dibromoethane, 1-bromo-2-chloroethane, 1,3-dibromopropane, bis(2-chloroethyl)amine hydrochloride, mechlorethamine hydrochloride, or tris(2-chlorethyl)amine hydrochloride.

In the disclosed polymers, the average number of connections from the polymerized amine monomers (or amine repeat units) to the rest of the polymer is typically above 2.05, and more commonly in the range from about 2 to about 6. For example the range can be from about 2 to about 2.5, about 2.05 to about 3, 2.05 to about 4, about 2.05 to about 5, about 2.5 to about 3, about 2.5 to about 4, about 2.5 to about 5, about 2.5 to about 6, about 3 to about 4, about 3 to about 5, about 3 to about 6, about 4 to about 5, about 4 to about 6, about 5 to about 6. Each “X” group in Structural Formulas (XIII)-(XXII) that is a covalent bond to another atom in the polymer is a “connection”. The average number of connections in a polymer is the total number of connections per total number of polymerized amine monomer (or repeat units). A “connection” is typically from a polymerized amine monomer (or amine repeat unit) to a crosslinking group. For example, when an “X” group connects to another atom in the polymer, the connection is typically to a crosslinking group.

The molecular weight of the disclosed polymers is not believed to be critical, provided that the molecular weight is large enough so that the polymer is not readily absorbed by the gastrointestinal tract. Typically the molecular weight is at least 1000. For example the molecular weight can be from about 1000 to about 5 million, about 1000 to about 3 million, about 1000 to about 2 million or about 1000 to about 1 million. Crosslinked polymers, however, are not generally characterized by molecular weight.

Physiologically acceptable salts of the disclosed polymers are also encompassed within the invention. “Physiologically acceptable” means suitable for pharmaceutical use. The term “salt” as used with reference to any of the disclosed phosphate binding polymers refers to protonization of the polymer into the form of a salt. For example, some or all of the nitrogen-bearing functional groups in the disclosed polymers may be protonated to create a positively charged nitrogen atom associated with a negatively charged counterion. In one embodiment, less than about 50%, for example, less than 30%, such as less than 20% or less than 10% of the amine groups in the disclosed polymers are protonated. In another embodiment 35% to 45% of the amines are protonated (e.g., approximately 40%).

“Physiologically acceptable salts” of the disclosed polymers are prepared from physiologically acceptable acids including inorganic acids and organic acids. Negatively charged counterions can be organic ions, inorganic ions, or a combination thereof. The inorganic ions suitable for use with embodiments of the invention include halide (especially chloride), carbonate, bicarbonate, sulfate, bisulfate, hydroxide, nitrate, persulfate and sulfite. Suitable organic ions include acetate, ascorbate, benzoate, citrate, dihydrogen citrate, hydrogen citrate, oxalate, succinate, tartrate, taurocholate, glycocholate, and cholate. Protonated polymers can optionally comprise two or more different negatively charged counterions.

As used herein, the term “optionally quaternarized” indicates that the designated amine group may optionally be bonded to a designated fourth group, yielding the corresponding positively charged ammonium group. An ammonium group is associated with a physiologically acceptable counteranion, as described above. Suitable counteranions are as provided above with reference to physiologically acceptable salts.

An “acyclic nitrogen atom” is a nitrogen atom that is not a ring atom of a heteroaryl or heterocyclic group.

The term “amine or amine group” includes primary, secondary and tertiary amines, as well as quaternary amines (ammonium groups).

An “alkyl group or alkyl”, as used herein, is a saturated straight chained or branched or cyclic hydrocarbon. Cyclic hydrocarbons are also referred to herein as “alicyclic groups”. Typically, straight chained or branched groups have from one to ten carbons, or more typically one to five carbons. Cyclic alkyl groups typically have three to eight ring carbon atoms. Examples of alkyl groups include, but are not limited to, methyl, ethyl, n-propyl, isopropyl, n-butyl, sec-butyl, tert-butyl, pentyl, iso-pentyl, neopentyl, hexyl, heptyl, octyl, nonyl, decyl, undecyl, dodecyl, cyclopropyl, cyclopentyl, cyclohexyl and the like. An alkyl group may be substituted with one or more substituents independently selected for each position.

The term “aryl group” may be used interchangeably with “aryl,” “aryl ring,” “aromatic group,” and “aromatic ring.” Aryl groups include carbocyclic aromatic groups, typically with six to fourteen ring carbon atoms (e.g., phenyl, naphthyl, and anthracyl groups). Aryl groups also include heteroaryl groups, which typically have five to fourteen ring atoms with one or more heteroatoms selected from nitrogen, oxygen and sulfur. A heteroaryl group can be monocyclic or a fused polycyclic aromatic ring systems in which a carbocyclic aromatic ring or heteroaryl ring is fused to one or more other heteroaryl rings. Examples of heteroaryl groups include furanyl, imidazolyl, isoxazolyl, oxadiazolyl, oxazolyl, pyrazolyl, pyrrolyl, pyridyl, pyrimidinyl, pyridazinyl, thiazolyl, triazolyl, tetrazolyl, thienyl, benzimidazolyl, benzothienyl, benzofuranyl, indolyl, quinolinyl, benzotriazolyl, benzothiazolyl, benzoxazolyl, benzimidazolyl, isoquinolinyl, indolyl, isoindolyl, or benzisoxazolyl. Preferably, the aryl group is a phenyl group.

A “heterocyclic group” is a non-aromatic mono or bicyclic group with three to twelve ring atoms. One, two or three of the ring atoms are heteroatoms selected from oxygen, nitrogen or sulfur. Monocyclic rings with three to eight ring atoms, one or two of which are oxygen, nitrogen or sulfur are more commonly used. Examples include morpholinyl, thiomorpholinyl, pyrrolidinyl, piperazinyl, piperidinyl, thiazolidinyl and oxazolinidyl.

A “carbocyclic ring” is ring in which the ring atoms are all carbons.

Optionally substituted alkyl, heterocyclic or aryl groups may carry one or more substituents which do not significantly adversely affect the phosphate binding ability of the polymers. Suitable substituents include amino, alkylamino, dialkylamino, aminocarbonyl, ammonium, dialkylammonium, trialkylammonium, halogen, alkyl, alkylaminocarbonyl, dialkylaminocarbonyl, alkylaminocarbonyloxy, dialkylaminocarbonyloxy, alkoxy, nitro, cyano, carboxy, alkoxycarbonyl, alkylcarbonyl, hydroxy, haloalkoxy, or haloalkyl. Preferred substituents include C₁-C₃ alkyl group, C₁-C₃ haloalkyl group, hydroxy, amino, alkylamino, dialkylamino, ammonium, dialkylammonium, trialkylammonium, halo, C₁-C₃ alkoxy or C₁-C₃ haloalkoxy.

The disclosed polymers can be used to remove target anions from a subject in need of such treatment. A “target anion” is an anion that is present at elevated levels in a subject and is causing or contributing to a pathological condition or disease. Examples of target anions include phosphate, bile acids, oxalate, and fatty acids.

The disclosed polymers are commonly used to treat subjects with elevated phosphate levels. Subjects with elevated phosphate levels include those with hyperphosphatemia, end stage renal disease, chronic kidney disease, hyperthyroidism, overmedication with phosphate salts, acromegaly, depressed renal synthesis of calcitriol, renal insufficiency, hypocalcemia, tetany due to hypocalcemia, ectopic calcification in soft tissues, and acute tissue destruction as occurs during rhabdomyolysis and treatment of malignancies.

As used herein a “subject” is a mammal, preferably a human, but can also be an animal in need of veterinary treatment, such as a companion animal (e.g., dogs, cats, and the like), a farm animal (e.g., cows, sheep, pigs, horses, and the like) or a laboratory animal (e.g., rats, mice, guinea pigs, and the like).

The disclosed polymers are also used to control the serum phosphate in subjects with elevated phosphate levels.

As used herein “controlling serum phosphate” means changing the serum level of phosphate towards a normal or near normal level, for example, towards a level that is within 10% of the normal level of a healthy subject.

As used herein a “patient” is a subject, typically a human subject.

An “effective amount” of a disclosed polymer is an amount that decreases the serum level of the target anion. Alternatively, an “effective amount” of the disclosed polymer is a quantity sufficient to achieve a therapeutic and/or prophylactic effect on a particular condition being treated, such as an amount which results in the prevention of or a decrease in the symptoms associated with the disease associated. The precise amount of the disclosed polymers that is administered to the individual will depend on the type and severity of the disease and on the characteristics of the individual, such as general health, age, sex, body weight and tolerance to drugs. The skilled artisan will be able to determine appropriate dosages depending on these and other factors. Typical dosages of polymers of the invention range from about 5 milligrams/day to about 10 grams/day, preferably from about 50 milligrams/day to about 9 grams/day, more preferably from about 1 gram/day to about 8 grams/day, even more preferably about 2 grams to about 7 grams, most preferably about 4 grams/day to about 6 grams/day. These dosages can be administered several times/day (e.g., 2, 3, 4 or 5 times/day) or once/day. The disclosed polymers can be administered, for example, at least four times per day, preferably with, before or after meals, at least three times per day with, before or after meals, at least twice per day with, before or after meals, at least once per day with, before or after meals, In one specific example, about 0.8-7.2 g (e.g., 2.4 g or 3.2 g per dose for 2-3 times per day, or 4.0 or 4.8 g per dose for 2-3 times per day, or 7.2 or 8.0 or 8.8 or 9.6 g per dose for once per day) of the disclosed polymers is administered per day.

Typically, the disclosed polymers can be administered before or after a meal, or with a meal. As used herein, “before” or “after” a meal is typically within two hours, preferably within one hour, more preferably within thirty minutes, most preferably within ten minutes of commencing or finishing a meal, respectively.

The disclosed polymers can be administered by any suitable route, but are typically administered orally, for example, in capsules, suspensions or tablets.

Still other embodiments of the invention are directed towards pharmaceutical compositions comprising at least one of the disclosed polymers or a pharmaceutically acceptable salt of the polymer, and a diluent of pharmaceutically acceptable carrier. The disclosed polymers may be lyophilized or dried under vacuum or oven before formulating. Optionally, one or more other therapeutic ingredients, including other phosphate binding agents, are included in such pharmaceutical compositions. The polymer may be any of the polymers described by embodiments of the invention herein.

The carriers of diluents are “acceptable” in the sense of being compatible with the other ingredients of the formulation and not deleterious to the recipient thereof. The formulations can conveniently be presented in unit dosage form and can be prepared by any suitable method known to the skilled artisan. The methods typically include the step of bringing into association the agent with the carrier or diluent which constitutes one or more accessory ingredients. In general, the formulations are prepared by uniformly and intimately bringing into association the disclosed polymer with the carriers and then, if necessary, dividing the product into unit dosages thereof.

Those skilled in the art will be aware that the amounts of the various components of the compositions of the invention to be administered in accordance with the method of the invention to a subject will depend upon those factors noted above. Such amounts may correspond with a dosage to be administered over a particular period of time to a subject (e.g., one or more tablets containing a single dose, or a sachet, slurry, food formulation, suspension, or syrup comprising a single dose).

The compositions of the invention can be formulated as a tablet, sachet, slurry, food formulation, troche, capsule, elixir, suspension, syrup, wafer, chewing gum or lozenge. A syrup formulation will generally consist of a suspension or solution of the disclosed polymer or salt in a liquid carrier, for example, ethanol, glycerine or water, with a flavoring or coloring agent. Where the composition is in the form of a tablet, one or more pharmaceutical carriers routinely used for preparing solid formulations can be employed. Examples of such carriers include magnesium stearate, starch, lactose and sucrose. Where the composition is in the form of a capsule, the use of routine encapsulation is generally suitable, for example, using the aforementioned carriers in a hard gelatin capsule shell. Where the composition is in the form of a soft gelatin shell capsule, pharmaceutical carriers routinely used for preparing dispersions or suspensions can be considered, for example, aqueous gums, celluloses, silicates or oils, and are incorporated in a soft gelatin capsule shell.

Though the above description is directed toward routes of oral administration of pharmaceutical compositions consistent with embodiments of the invention, it is understood by those skilled in the art that any mode of administration, vehicle or carrier conventionally employed and which is inert with respect to the disclosed polymer may be utilized for preparing and administering the compositions. Illustrative of such methods, vehicles and carriers are those described, for example, in Remington's Pharmaceutical Sciences, 18^{th} ed. (1990), the disclosure of which is incorporated herein by reference.

The disclosed polymers can be administered or formulated alone or in combination with other pharmaceutically active agents, e.g., other agents which bind phosphate or other target anions, agents which inhibit phosphate transport, alkaline phosphatase inhibitors, HMG-CoA reductase inhibitors, cholesteroal absorption inhibitors and bile acid sequestrants.

An agent which binds phosphate and can advantageously be used in combination with the disclosed polymers is a pharmaceutically acceptable magnesium compound (see, for example, U.S. 60/734,593, the entire teachings of which are incorporated herein by reference), which refers to a compound comprising a magnesium cation and which does not cause unacceptable side effects at the dosages which are being administered. The pharmaceutically acceptable magnesium compound can be water-soluble or water-insoluble. Preferred pharmaceutically acceptable magnesium compounds have a high weight percentage of magnesium, and/or have a high density. These magnesium compounds can minimize daily dose volume. Examples of magnesium compounds suitable for the invention include magnesium oxide, magnesium hydroxide, magnesium halides (e.g., magnesium fluoride, magnesium chloride, magnesium bromide and magnesium iodide), magnesium alkoxides (e.g., magnesium ethoxide and magnesium isopropoxide), magnesium carbonate, magnesium bicarbonate, magnesium formate, magnesium acetate, magnesium trisilicates, magnesium salts of organic acids, such as fumaric acid, maleic acid, acrylic acid, methacrylic acid, itaconic acid and styrenesulfonic acid, and a combination thereof. When referring to any of these magnesium compounds, it is to be understood that mixtures, polymorphs and solvates thereof are encompassed.

Other phosphate binders include pharmaceutically acceptable lanthanum, calcium, aluminum, iron and zinc salts (see, for example, U.S. 60/640,643, the entire teachings of which are incorporated herein by reference), such as acetates, carbonates, oxides, hydroxides, citrates, alginates, and ketoacids. Calcium salts, including calcium carbonate, acetate (such as PhosLo® calcium acetate tablets), citrate, alginate, and ketoacids, have been utilized for phosphate binding. The ingested calcium combines with phosphate to form insoluble calcium phosphate salts such as Ca₃(PO₄)₂, CaHPO₄, or Ca(H₂PO₄)₂. Aluminium-based phosphate binders, such as Amphojel® aluminium hydroxide gel, have also been used for treating hyperphosphatemia. These compounds complex with intestinal phosphate to form highly insoluble aluminum phosphate; the bound phosphate is unavailable for absorption by the patient. More recently lanthanide salts have been used. The most commonly used lanthanide salt, lanthanum carbonate (Fosrenol®) behaves similarly to calcium carbonate. Other compositions which may be used with the disclosed polymers of the present invention include other types of phosphate-binding polymers (e.g., sevelamer hydrochloride as described in U.S. Pat. No. 5,667,775, which is hereby incorporated herein by reference in its entirety).

HMG-CoA reductase inhibitors (e.g. statins) include lovastatin (mevinolin) (e.g., Altocor® and Mevacoro®) and related compounds; pravastatin (e.g., Pravachol®, Selektine®, and Lipostat®) and related compounds; simvastatin (e.g., Zocor®) and related compounds. Other HMG-CoA reductase inhibitors which can be employed in the present invention include fluvastatin (e.g., Lescol®; cerivastatin (e.g., Baycol® and Lipobay®); atorvastatin (e.g., Zarator® and Lipitor®); pitavastatin; rosuvastatin (visastatin)(e.g., Crestor®); quinoline analogs of mevalonolactone and derivatives thereof (see U.S. Pat. No. 5,753,675); pyrazole analogs of mevalonolactone derivatives (see U.S. Pat. No. 4,613,610); indene analogs of mevalonolactone derivatives (see WO 86/03488); 6-[2-(substituted-pyrrol-1-yl)-alkyl)pyran-2-ones and derivatives thereof (see U.S. Pat. No. 4,647,576); imidazole analogs of mevalonolactone (see WO 86/07054); 3-hydroxy-4(dihydroxooxophosphorio) butanoic acid derivatives (see French Patent No. 5,596,393); naphthyl analogs of mevalonolactone (see U.S. Pat. No. 4,686,237); octahydronaphthalenes (see U.S. Pat. No. 4,499,289); and quinoline and pyridine derivatives (see U.S. Pat. Nos. 5,506,219 and 5,691,322). A statin, such as atorvastatin, fluvastatin, lovastatin, pravastatin, simvastatin, rosuvastatin, cerivastatin and pitavastatin, is preferred.

An example of a cholesterol absorption inhibitor is ezetimibe.

Examples of phosphate transport inhibitors are found in co-pending U.S. Application Nos. 2004/0019113 and 2004/0019020 and WO 2004/085448, the entire teachings of each of these are incorporated herein by reference.

Examples of alkaline phosphatase inhibitors include orthophosphate, arsenate, L-phenylalanine, L-homoarginine, tetramisole, levamisole, L-p-Bromotetramisole, 5,6-Dihydro-6-(2-naphthyl)imidazo-[2,1-b]thiazole (napthyl) and derivatives thereof. The preferred inhibitors include, but are not limited to, levamisole, bromotetramisole, and 5,6-Dihydro-6-(2-naphthyl)imidazo-[2,1-b]thiazole and derivatives thereof.

Examples of bile acid sequestrants include colesevelam, cholestyramine, and colestipol.

The invention is described by the following examples which are not intended to be limiting in any way.

### EXEMPLIFICATION

### Example 1

### Synthesis of tris(2-aminoethyl)amine/epichlorohydrin (1:1) condensation Polymer

To a solution of tris(2-aminoethyl)amine (22.42 mL) in methanol (35 mL) under nitrogen was added epichlorohydrin (11.73 mL). Upon addition of the epichlorohydrin the reaction exothermed to 74° C. After the exotherm subsided, the solution was heated to reflux (temperature setting of 75° C.) for 24 h. During this period the reaction turned from a solution to a block gel. After cooling to room temperature, the block gel was broken into small pieces with a potato masher, and suspended in methanol (500 mL). After stirring for at least 30 minutes, the suspension was filtered. The polymer was similarly washed twice more with methanol. The polymer was then suspended in deionized water (500 mL), stirred for at least 30 minutes, and filtered. The polymer was suspended again in deionized water (500 mL), stirred for at least 30 minutes. The pH of the suspension was adjusted to 7 with the addition of concentrated hydrochloric acid. The suspension was filtered and the polymer was dried in a forced air oven at 60° C. The dried polymer (rubbery solid) was suspended in deionized water (3 L) and stirred for 1 h. The pH of the suspension was adjusted to 1 with the addition of concentrated HCl. The suspension was filtered and the wet polymer (431.65 g) was dried in a forced air oven at 60° C. to afford 17.25 g of a solid which was ground to a powder in a coffee mill.

Polymers 1-26 were prepared similarly to Example 1 using the reactants and reaction conditions as listed in Table 1.

TABLE 1

| Polymer | | | | | Temp | Time | | Swelling |
|---|---|---|---|---|---|---|---|---|
| No. | amine | electrophile | base | solvent | (o C.) | (in hour) | yield | (mL/g) |
| 1 | tris(2-aminoethyl)amine, | epichlorohydrin, | | toluene, 200 mL; | 95 | 24 | 17.39 g | 12.7 |
| | 11.88 mL | 9.32 mL | | water, 40 mL | | | | |
| 2 | tris(2-aminoethyl)amine, | epichlorohydrin, | | toluene, 200 mL; | 95 | 24 | 22.54 g | 3 |
| | 11.88 mL | 12.44 mL | | water, 40 mL | | | | |
| 3 | tris(2-aminoethyl)amine, | epichlorohydrin, | | methanol, 35 mL | 75 | 24 | 17.25 g | 24.02 |
| | 22.42 mL | 11.73 mL | | | | | | |
| 4 | tris(2-aminoethyl)amine, | epichlorohydrin, | | methanol, 35 mL | 75 | 24 | 36.76 g | 2.6 |
| | 22.42 mL | 17.6 mL | | | | | | |
| 5 | tris(2-aminoethyl)amine, | epichlorohydrin, | | methanol, 35 mL | 75 | 24 | 46.12 | 1.44 |
| | 22.42 mL | 23.47 mL | | | | | | |
| 6 | tris(2-aminoethyl)amine, | epichlorohydrin, | | toluene, 30 mL; | 40 | 24 | 25.56 g | 10.27 |
| | 22.42 mL | 11.73 mL | | water, 5 mL | | | | |
| 7 | tris(2-aminoethyl)amine, | 1,2-dibromoethane, | | methanol, 25 mL | 75 | 24 | 26.13 g | 8.36 |
| | 22.43 mL | 12.93 mL | | | | | | |
| 8 | tris(2-aminoethyl)amine, | 1,2-dibromoethane, | | methanol, 25 mL | 40 | 24 | 30.72 g | 3.97 |
| | 22.43 mL | 19.39 mL | | | | | | |
| 9 | tris(2-aminoethyl)amine, | 1,2-dibromoethane, | | methanol, 25 mL | 40 | 24 | 29.11 g | 2.52 |
| | 22.43 mL | 25.85 mL | | | | | | |
| 10 | tris(2-aminoethyl)amine, | 1-bromo-2-chloroethane, | | methanol, 25 mL | 40 | 24 | 22.29 | 28.12 |
| | 22.43 mL | 12.44 mL | | | | | | |
| 11 | tris(2-aminoethyl)amine, | 1,3-dibromopropane, | | methanol, 25 mL | 40 | 24 | 33.07 g | 11.63 |
| | 22.43 mL | 15.32 mL | | | | | | |
| 12 | tris(2-aminoethyl)amine, | 1,2-dibromoethane, | | methanol, 25 mL | 40 | 24 | 25.48 g | 10.96 |
| | 22.43 mL | 12.93 mL | | | | | | |
| 13 | triethylenetetramine, | 1,2-dibromoethane, | | methanol, 25 mL | 60 | 24 | 1.02 g | |
| | 22.54 mL | 12.93 mL | | | | | | |
| 14 | tetraethylenepentamine, | 1,2-dibromoethane, | | methanol, 25 mL | 60 | 24 | 3.24 g | |
| | 28.16 mL | 12.93 mL | | | | | | |
| 15 | pentaethylenehexamine, | 1,2-dibromoethane, | | methanol, 25 mL | 60 | 24 | 4.02 g | |
| | 34.86 mL | 12.93 mL | | | | | | |
| 16 | N-boc-ethylenediamine, | 1,2-dibromoethane, | | methanol, 25 mL | 60 | 24 | 1.02 g | |
| | 24 g | 12.93 mL | | | | | | |
| 17 | tris(methylamino)ethyl- | 1,2-dibromoethane, | | methanol, 25 mL | 60 | 24 | 5.40 g | |
| | amine, 31.53 mL | 12.93 mL | | | | | | |
| 18 | tris(2-aminoethyl)amine, | bis(2-chloroethyl)amine | | methanol, 25 mL | 60 | 24 | 24.65 g | 4.25 |
| | 22.43 mL | hydrochloride, | | | | | | |
| | | 26.77 g | | | | | | |
| 19 | tris(2-aminoethyl)amine, | tris(2-chloroethyl)amine | | methanol, 25 mL | 60 | 24 | 25.91 g | 4.16 |
| | 22.43 mL | hydrochloride, | | | | | | |
| | | 28.88 g | | | | | | |
| 20 | pentaethylenehexamine, | 1,2-dibromoethane, | Na2CO3, | methanol, 35 mL | 60 | 24 | 12.36 g | |
| | 34.86 mL | 12.93 mL | 15.9 g | | | | | |
| 21 | pentaethylenehexamine, | 1,3-dibromopropane, | | methanol, 35 mL | 60 | 24 | 7.75 g | |
| | 34.86 mL | 15.32 mL | | | | | | |
| 22 | pentaethylenehexamine, | 1,3-dibromopropane, | | methanol, 35 mL | 60 | 24 | 10.45 g | |
| | 34.86 mL | 22.98 mL | | | | | | |
| 23 | pentaethylenehexamine, | 1,3-dibromopropane, | | methanol, 35 mL | 60 | 24 | 62.88 g | 8.94 |
| | 34.86 mL | 30.64 mL | | | | | | |
| 24 | 1,3,5-triamino | epichlorohydrin | NaOH | Water, 56 mL | 60 | 18 | 10.43 g | 12.47 |
| | cyclohexane•3HBr (14 g) | (5.9 mL) | | | | | | |
| 25 | Bis(dipropylenetriamino) | epichlorohydrin | | Water, 60 mL | 60 | 18 | 22.42 | 6.98 |
| | tetramethylbenzene | ((3.02 mL) | | | | | | |
| | (15 g) | | | | | | | |
| 26 | 1,2,4,5-tetrakis(amino- | epichlorohydrin, | NaOH | Water, 120 mL | 60 | 18 | 35.48 | 1.98 |
| | methyl) benzene•4HCl | 13.76 mL | | | | | | |
| | (30 g) | | | | | | | |

### Example 2

### Effects of Amine Condensation Polymers for Reducing Urinary Phosphate Levels

House male Sprague Dawley (SD) rats were used for the experiments. The rats were placed singly in wire-bottom cages, fed with Purina 5002 diet, and allowed to acclimate for at least 5 days prior to experimental use.

To establish baseline phosphorus excretion, the rats were placed in metabolic cages for 48 hours. Their urine was collected and its phosphorus content analyzed with a Hitachi analyzer to determine phosphorus excretion in mg/day. Any rats with outlying values were excluded; and the remainder of the rats were distributed into groups.

Purina 5002 was used as the standard diet. The polymer being tested was mixed with Purina 5002 to result in a final concentration 0.5% by weight. Cellulose at 0.5% by weight was used as a negative control. For each rat, 200 g of diet was prepared.

Each rat was weighed and placed on the standard diet. After 4 days the standard diet was replaced with the treatment diet (or control diet for the control group). On days 5 and 6, urine samples from the rats at 24 hours (+/−30 minutes) were collected and analyzed. The test rats were again weighed, and any weight loss or gain was calculated. Any remaining food was also weighed to calculate the amount of food consumed per day. A change in phosphorus excretion relative to baseline and cellulose negative control was calculated using Excel program. A summary of comparison of the amounts of urinary phosphate obtained from the test rats is shown in Table 2.

TABLE 2
In Vivo Phosphate Sequestration Data

| Polymer ID (as referred | Polymer Dose | Urinary Phosphate Excretion |
|---|---|---|
| to in Table 1) | (Wt % of Diet) | % of Negative Control* |
| Example 3 | 0.50 | 60.7 |
| Example 25 | 0.5 | 73.8 |
| Example 26 | 0.5 | 98.2 |

*Negative control has a value of 100%

While this invention has been particularly shown and described with references to preferred embodiments thereof, it will be understood by those skilled in the art that various changes in form and details may be made therein without departing from the scope of the invention encompassed by the appended claims.

What is claimed is:

1. A polymer or physiologically acceptable salt thereof comprising a polymerized multifunctional amine monomer wherein the amine monomer comprises at least two amine groups and at least two acyclic nitrogen atoms that are connected through a —CH₂CH₂— group, provided that the amine monomer is not ethylenediamine or diethylenetriamine.

2. The polymer of claim 1 wherein the amine monomer comprises at least three nitrogen atoms.

3. The polymer of claim 1 wherein the amine monomer is represented by the following structural formula: 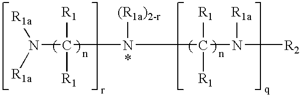
wherein:
each R₁, independently, is H or an optionally substituted alkyl group or an optionally substituted aryl group, or forms together with an R₁ bonded to an adjacent carbon or nitrogen atom and their intervening atoms an optionally substituted alicyclic, aromatic, or heterocyclic group;
each R₁ₐ is independently R₁ or 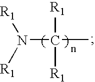
R₂ is R^{1a} or a group represented by the following structural formula: 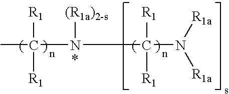
each nitrogen atom designated with “*” is optionally quarternized with R^{1a};
q is 0 or an integer from 1 to 10; r and s are 0, 1, or 2 with the proviso that the sum of r, s and q is greater than 1; and
each n, independently is an integer from 2 to 10 with the proviso that at least one n is 2.

4. The polymer of claim 3, wherein the amine monomer is represented by the following structural formula:

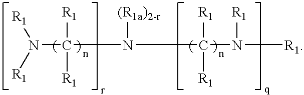

5. The polymer of claim 3, wherein the amine monomer is represented by the following structural formula:

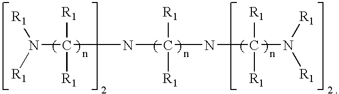

6. The polymer of claim 5, wherein the amine monomer is represented by the following structural formula:

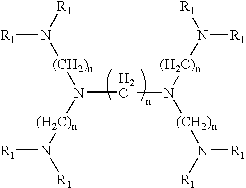

7. The polymer of claim 1 wherein the amine monomer is represented by the following structural formula: 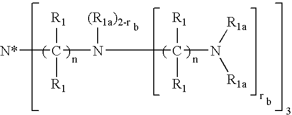
wherein each R₁, independently, is H or an optionally substituted alkyl group or an optionally substituted aryl group, or forms together with an R₁ bonded to an adjacent carbon or nitrogen atom and their intervening atoms an optionally substituted alicyclic, aromatic, or heterocyclic group;
each R₁ₐ is independently R₁ or 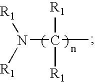
each nitrogen atom designated with “*” is optionally quarternized with R₁ₐ;
each r_{b}, independently, is 0, 1, or 2; and
each n, independently, is an integer from 2 to 10 with the proviso that at least one n is 2.

8. The polymer of claim 7, wherein the amine monomer is represented by the following structural formula;

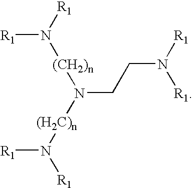

9. The polymer of claim 1, wherein the amine monomer is represented by the following structural formula: 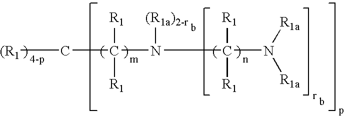
wherein each R₁, independently, is H or an optionally substituted alkyl group or an optionally substituted aryl group, or forms together with an R₁ bonded to an adjacent carbon or nitrogen atom and their intervening atoms an optionally substituted alicyclic, aromatic, or heterocyclic group;
each R₁ₐ is independently R₁ or 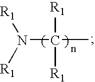
p is 1, 2, 3, or 4; each r_{b}, independently, is 0, 1, or 2 with the proviso that r_{b} is 1 or 2 if p is equal to 1;
each m, independently, is 0 or an integer from 1 to 10; and
each n, independently, is an integer from 2 to 10 with the proviso that at least one n is 2.

10. The polymer of claim 9 wherein the amine monomer is represented by the following structural formula:

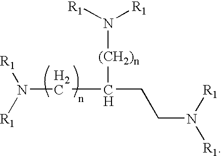

11. The polymer of claim 1, wherein the amine monomer is represented by the following structural formula: 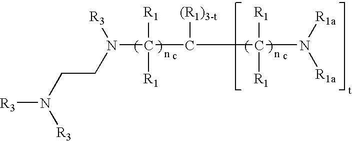
wherein each R₁, independently, is H or an optionally substituted alkyl group or an optionally substituted aryl group, or forms together with an R₁ bonded to an adjacent carbon or nitrogen atom and their intervening atoms an optionally substituted alicyclic, aromatic, or heterocyclic group;
each R^{1a} is independently R¹ or 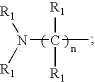
each R₃, independently, is H, 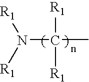
or an optionally substituted alkyl group or an optionally substituted aryl group;
each t, independently, is 0, 1, 2, or 3; and
each n_{c}, independently, is 0 or an integer from 1 to 10.
each n is integer from 2 to 10.

12. (canceled)

13. A polymer or physiologically acceptable salt thereof comprising an amine repeat unit, wherein the repeat unit comprises at least two amine groups and at least two acyclic nitrogen atoms that am connected through a —CH₂CH₂— group, provided that the repeat unit is not —NHCH₂CH₂NH—, —NHCH₂CH₂NHCH₂CH₂NH—, —NHCH₂CH₂(N—)CH₂CH₂NH—, or —NHCH₂CH₂(N—)CH₂CH₂NH₂.

14. The polymer of claim 13 wherein the amine repeat unit is represented by the following structural formula: 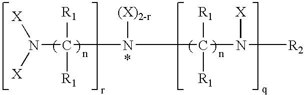
wherein each R¹, independently, is H or an optionally substituted alkyl group or an optionally substituted aryl group, or forms together with an R₁ bonded to an adjacent carbon or nitrogen atom and their intervening atoms an optionally substituted alicyclic, aromatic, or heterocyclic group;
two or more of the groups represented by X are each a covalent bond to another atom in the polymer, and the remainder of the groups represented by X are R₁;
R₂ is X or a group represented by the following structural formula: 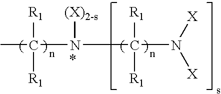
each nitrogen atom designated with “*” is optionally quarternized with R₁ or 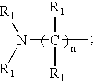
q is 0 or an integer from 1 to 10; r and s are 0, 1, or 2 with the proviso that the sum of r, s and q is greater than 1; and
each n, independently, is an integer from 2 to 10 with the proviso that at least one n is 2.

15. The polymer of claim 14, wherein the amine repeat unit is represented by the following structural formula:

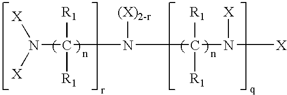

16. The polymer of claim 14, wherein the amine repeat unit is represented by the following structural formula:

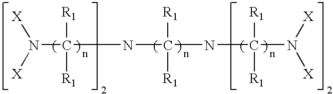

17. The polymer of claim 16, wherein the amine amine repeat unit is represented by the following structural formula:

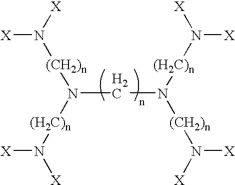

18. The polymer of claim 13 wherein the amine repeat unit is represented by the following structural formula: 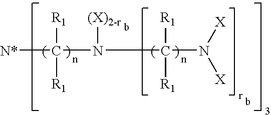
wherein each R₁, independently, is H or an optionally substituted alkyl group or an optionally substituted aryl group, or forms together with an R₁ bonded to an adjacent carbon or nitrogen atom and their intervening atoms an optionally substituted alicyclic, aromatic, or heterocyclic group;
two or more of the groups represented by X are each a covalent bond to another atom in the polymer, and the remainder of the groups represented by X are R₁;
each r_{b}, independently, is 0, 1, or 2; and
the nitrogen atom designated with “*” is optionally quarternized with R₁ or 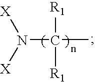
each n, independently, is an integer from 2 to 10 with the proviso that at least one n is 2.

19. The polymer of claim 18, wherein the amine repeat unit is represented by the following structural formula:

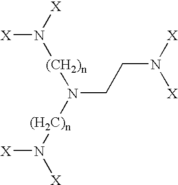

20. The polymer of claim 13 wherein the amine repeat unit is represented by the following structural formula: 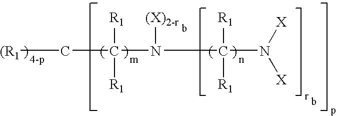
wherein each R₁, independently, is H or an optionally substituted alkyl group or an optionally substituted aryl group, or forms together with an R₁ bonded to an adjacent carbon or nitrogen atom and their intervening atoms an optionally substituted alicyclic, aromatic, or heterocyclic group;
two or more of the groups represented by X are each a covalent bond to another atom in the polymer, and the remainder of the groups represented by X are R₁;
p is 1, 2, 3, or 4; each r_{b}, independently, is 0, 1, or 2 with the proviso that r_{b} is 1 or 2 if p is equal to 1;
each m, independently, is 0 or an integer from 1 to 10; and
each n, independently, is an integer from 2 to 10 with the proviso that at least one n is 2.

21. The polymer of claim 20 wherein the amine repeat unit is represented by the following structural formula: 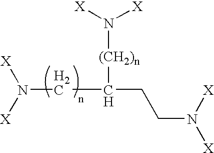
wherein each n_{b}, independently, is an integer from 2 to 10.

22. The polymer of claim 13 wherein the amine repeat unit is represented by the following structural formula: 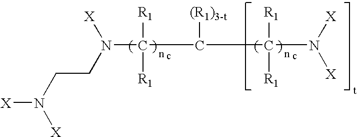
wherein each R₁, independently, is H or an optionally substituted alkyl group or an optionally substituted aryl group, or forms together with an R₁ bonded to an adjacent carbon or nitrogen atom and their intervening atoms an optionally substituted alicyclic, aromatic, or heterocyclic group;
two or more of the groups represented by X are each a covalent bond to another atom in the polymer and the remainder of the groups represented by X are R₁;
each t, independently, is 0, 1, 2, or 3; and
each n_{c}, independently, is 0 or an integer from 1 to 10.

23. (canceled)

24. The polymer of claim 1 wherein the amine monomer is selected from the group consisting of tris(2-aminoethyl)amine, triethylenetetramine, tetraethylenepentamine, pentaethylenehexamine, N-boc-ethylenediamine, tris[(methylamino)ethyl]amine and N,N,N′,N′-tetrakis(3-aminopropyl)1,2-diaminoethane; and the amine repeat unit is selected from the group consisting of polymerized tris(2-aminoethyl)amine, triethylenetetramine, tetraethylenepentamine, pentaethylenehexamine, N-boc-ethylenediamine, tris[(methylamino)ethyl]amine, and N,N,N′,N′-tetrakis(3-aminopropyl) 1,2-diaminoethane.

25. The polymer of claim 1 wherein the polymer comprises a first and a second amine monomer wherein the first amine monomer is different from the second amine monomer; or wherein the polymer comprises a first and a second amine repeat unit wherein the first amine repeat unit is different from the second amine repeat unit.

26. The polymer of claim 1, wherein the polymer is crosslinked with a multifunctional crosslinking group.

27. The polymer of claim 26 wherein the multifunctional crosslinking group is the residue of a multifunctional crosslinking agent comprising two or more electrophilic groups.

28. The polymer of claim 27 wherein the electrophilic group is selected from the group consisting of a halide, epoxide, acrylate, arylsulfonate and alkylsulfonate.

29. The polymer of claim 26 wherein the multifunctional crosslinking group is the residue of a multifunctional crosslinking agent selected from the group consisting of a dihaloalkane, haloalkyloxirane, alkyloxirane sulfonate, di(haloalkyl)amine, trihaloalkyl)amine, diepoxide, triepoxide, tetraepoxide, bis(halomethyl)benzene, tri(halomethyl)benzene) and tetra(halomethyl)benzene.

30. The polymer of claim 29 wherein the multifunctional crosslinking agent is selected from the group consisting of epichlorohydrin, epibromohydrin, (iodomethyl)oxirane, glycidyl tosylate, glycidyl 3-nitrobenzenesulfonate, 4-tosyloxy-1,2-epoxybutane, bromo-1,2-epoxybutane, 1,2-dibromoethane, 1-bromo-2-chloroethane, 1,3-dibromopropane, bis(2-chloroethyl)amine, tris(2-chloroethyl)amine, and bis(2-chloroethyl)methylamine, 1,3-butadiene diepoxide, 1,5-hexadiene diepoxide, diglycidyl ether, 1,2,7,8-diepoxyoctane, 1,2,9,10-diepoxydecane, ethylene glycol diglycidyl ether, propylene glycol diglycidyl ether, 1,4-butanediol diglycidyl ether, glycerol diglycidyl ether, 1,3-diglycidyl) glyceryl ether, N,N-diglycidylaniline, neopentyl glycol diglycidyl ether, diethylene glycol diglycidyl ether, 1,4-bis(glycidyloxy)benzene, resorcinol digylcidyl ether, 1,6-hexanediol diglycidyl ether, trimethylolpropane diglycidyl ether, 1,4-cyclohexanedimethanol diglycidyl ether, 1,3-bis-(2,3-epoxypropyloxy)-2-(2,3-dihydroxypropyloxy)propane, 1,2-cyclohexanedicarboxylic acid diglycidyl ester, 2,2′-bis(glycidyloxy)diphenylmethane, bisphenol F diglycidyl ether, 1,4-bis(2′,3′-epoxypropyl)perfluoro-n-butane, 2,6-di(oxiran-2-ylmethyl)-1,2,3,5,6,7-hexahydropyrrolo[3,4-f]isoindol-1,3,5,7-tetraone, bisphenol A diglycidyl ether, ethyl 5-hydroxy-6,8-di(oxiran-2-ylmethyl)-4-oxo-4h-chromene-2-carboxylate, bis[4-(2,3-epoxy-propylthio)phenyl]-sulfide, 1,3-bis(3-glycidoxypropyl)tetramethyldisiloxane, 9,9-bis[4-(glycidyloxy)phenyl]fluorene, triepoxyisocyanurate, glycerol triglycidyl ether, N,N-diglycidyl-4-glycidyloxyaniline, isocyanuric acid (S,S,S)-triglycidyl ester, isocyanuric acid (R,R,R)-triglycidyl ester, triglycidyl isocyanurate, trimethylolpropane triglycidyl ether, glycerol propoxylate triglycidyl ether, triphenylolmethane triglycidyl ether, 3,7,14-tris[[3-(epoxypropoxy)propyl]dimethylsilyloxy]-1,3,5,7,9,11,14-heptacyclopentyltricyclo[7,3,3,15,11]heptasiloxane, 4,4′-methylenebis(N,N-diglycidylaniline), bis(halomethyl)benzene, bis(halomethyl)biphenyl and bis(halomethyl)naphthalene.

31. The polymer of claim 27 wherein the amine monomer is selected from the group consisting of tris(2-aminoethyl)amine, triethylenetetramine, tetraethylenepentamine, pentaethylenehexamine, N-boc-ethylenediamine, tris[(methylamino)ethyl]amine and N,N,N′,N′-tetrakis(3-aminopropyl)1,2-diaminoethane, and wherein the crosslinking agent is selected from the group consisting of epichlorohydrin, 1,2-dibromoethane, 1-bromo-2-chloroethane, 1,3-dibromopropane, bis(2-chloroethyl)amine hydrochloride, mechlorethamine hydrochloride, and tris(2-chlorethyl)amine hydrochloride; and the amine repeat unit is selected from the group consisting of polymerized tris(2-aminoethyl)amine, triethylenetetramine, tetraethylenepentamine, pentaethylenehexamine, N-boc-ethylenediamine, tris[(methylamino)ethyl]amine and N,N,N′,N′-tetrakis(3-aminopropyl)1,2-diaminoethane, and wherein the crosslinking agent is selected from the group consisting of epichlorohydrin, 1,2-dibromoethane, 1-bromo-2-chloroethane, 1,3-dibromopropane, bis(2-chloroethyl)amine hydrochloride, mechlorethamine hydrochloride, and tris(2-chlorethyl)amine hydrochloride.

32. The polymer claim 3 wherein each R₁, independently, is H or an alkyl group optionally substituted with —OH, alkoxy, halogen, or a phenyl or pyridyl group, wherein the phenyl and pyridyl groups are optionally substituted with —H, alkoxy, halogen, haloalkyl or haloalkoxy.

33. The polymer of claim 11 wherein each R₃, independently, is H or an alkyl group optionally substituted with —H, alkoxy, halogen, or a phenyl or pyridyl group, wherein the phenyl and pyridyl groups are optionally substituted with —OH, alkoxy, halogen, haloalkyl or haloalkoxy.

34. The polymer of claim 3 wherein each n is equal to 2.

35-44. (canceled)

45. A polymer or physiologically acceptable salt thereof comprising a polymerized multifunctional amine monomer represented by the following structural formula: 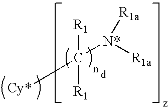
(Cy) is a C₄-C₁₀ saturated or unsaturated carbocyclic ring that is optionally substituted;
z is 2, 3 or 4;
each R₁, independently, is H or an optionally substituted alkyl group or an optionally substituted aryl group, or forms together with an R₁ bonded to an adjacent carbon or nitrogen atom and their intervening atoms an optionally substituted alicyclic, aromatic, or heterocyclic group;
each R₁, independently, is R¹, 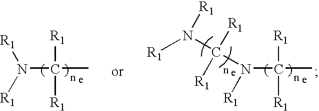
the nitrogen atom designated with “*” is optionally quarternized with R₁ₐ; and
each n_{d}, independently, is 0 or is an integer from 1 to 10 and each n_{c} is an integer from 2 to 10.

46. (canceled)

47. The polymer of claim 45 wherein (Cy) is cyclohexyl Or phenyl.

48. The polymer of claim 45 wherein each R₁, independently, is H or an alkyl group optionally substituted with —OH, alkoxy, halogen, or a phenyl or pyridyl group, wherein the phenyl and pyridyl groups are optionally substituted with —OH, alkoxy, halogen, haloalkyl or haloalkoxy.

49. The polymer of claim 45 wherein each n is equal to 2.

50. The polymer of claim 44, wherein the polymer is crosslinked with a crosslinking group.

51. The polymer of claim 49 wherein the amine repeat unit is selected from the group consisting of polymerized 1,3,5-tris(aminomethyl)cyclohexane and 1,2,4,5-tetrakis(aminomethyl)benzene, and the crosslinker is selected from the group consisting of epichlorohydrin, epibromohydrin, (iodomethyl)oxirane, glycidyl tosylate, glycidyl 3-nitrobenzenesulfonate, 4-tosyloxy-1,2-epoxybutane, bromo-1,2-epoxybutane, 1,2-dibromoethane, 1-bromo-2-chloroethane, 1,3-dibromopropane, bis(2-chloroethyl)amine, tris(2-chloroethyl)amine, bis(2-chloroethyl)methylamine, 1,3-butadiene diepoxide, 1,5-hexadiene diepoxide, diglycidyl ether, 1,2,7,8-diepoxyoctane, 1,2,9,10-diepoxydecane, ethylene glycol diglycidyl ether, propylene glycol diglycidyl ether, 1,4-butanediol diglycidyl ether, glycerol diglycidyl ether, 1,3-diglycidyl glyceryl ether, N,N-diglycidylaniline, neopentyl glycol diglycidyl ether, diethylene glycol diglycidyl ether, 1,4-bis(glycidyloxy)benzene, resorcinol digylcidyl ether, 1,6-hexanediol diglycidyl ether, trimethylolpropane diglycidyl ether, 1,4-cyclohexanedimethanol diglycidyl ether, 1,3-bis-(2,3-epoxypropyloxy)-2-(2,3-dihydroxypropyloxy)propane, 1,2-cyclohexanedicarboxylic acid diglycidyl ester, 2,2′-bis(glycidyloxy)diphenylmethane, bisphenol F diglycidyl ether, 1,4-bis(2′,3′-epoxypropyl)perfluoro-n-butane, 2,6-di(oxiran-2-ylmethyl)-1,2,3,5,6,7-hexahydropyrrolo[3,4-f]isoindol-1,3,5,7-tetraone, bisphenol A diglycidyl ether, ethyl 5-hydroxy-6,8-di(oxiran-2-ylmethyl)-4-oxo-4h-chromene-2-carboxylate, bis[4-(2,3-epoxy-propylthio)phenyl]-sulfide, 1,3-bis(3-glycidoxypropyl)tetramethyldisiloxane, 9,9-bis[4-glycidyloxy)phenyl]fluorene, triepoxyisocyanurate, glycerol triglycidyl ether, N,N-diglycidyl-4-glycidyloxyaniline, isocyanuric acid (S,S,S)-triglycidyl ester, isocyanuric acid (R,R,R)-triglycidyl ester, triglycidyl isocyanurate, trimethylolpropane triglycidyl ether, glycerol propoxylate triglycidyl ether, triphenylolmethane triglycidyl ether, 3,7,14-tris[[3-(epoxypropoxy)propyl]dimethylsilyloxy]-1,3,5,7,9,11,14-heptacyclopentyltricyclo[7,3,3,15,11]heptasiloxane, 4,4′-methylenebis(N,N-diglycidylaniline).

52-59. (canceled)

60. The polymer of claim 13 wherein the amine monomer is selected from the group consisting of tris(2-aminoethyl)amine, triethylenetetramine, tetraethylenepentamine, pentaethylenehexamine, N-boc-ethylenediamine, tris[(methylamino)ethyl]amine and N,N,N′,N′-tetrakis(3-aminopropyl) 1,2-diaminoethane; and the amine repeat unit is selected from the group consisting of polymerized tris(2-aminoethyl)amine, triethylenetetramine, tetraethylenepentaamine, pentaethylenehexamine, N-boc-ethylenediamine, tris[(methylamino)ethyl]amine, and N,N,N′,N′-tetrakis(3-aminopropyl)1,2-diaminoethane.

61. The polymer of claim 13, wherein the polymer is crosslinked with a multifunctional crosslinking group.

62. The polymer claim 14 wherein each R₁, independently, is H or an alkyl group optionally substituted with —OH, alkoxy, halogen, or a phenyl or pyridyl group, wherein the phenyl and pyridyl groups are optionally substituted with —OH, alkoxy, halogen, haloalkyl or haloalkoxy.

63. The polymer of claim 14 wherein each n is equal to 2.

64. The polymer claim 14 wherein each R₁, independently, is H or an alkyl group optionally substituted with —H, alkoxy, halogen, or a phenyl or pyridyl group, wherein the phenyl and pyridyl groups are optionally substituted with —OH, alkoxy, halogen, haloalkyl or haloalkoxy.

65. The polymer claim 15 wherein each R₁, independently, is H or an alkyl group optionally substituted with —OH, alkoxy, halogen, or a phenyl or pyridyl group, wherein the phenyl and pyridyl groups are optionally substituted with —OH, alkoxy, halogen, haloalkyl or haloalkoxy.

66. The polymer claim 20 wherein each R₁, independently, is H or an alkyl group optionally substituted with —OH, alkoxy, halogen, or a phenyl or pyridyl group, wherein the phenyl and pyridyl groups are optionally substituted with —OH, alkoxy, halogen, haloalkyl or haloalkoxy.

67. A pharmaceutical composition comprising:
a) a polymer or physiologically acceptable salt thereof comprising a polymerized multifunctional amine monomer wherein the amine monomer comprises at least two amine groups and at least two acyclic nitrogen atoms that are connected through a CH₂CH₂— group, provided that the amine monomer is not ethylenediamine or diethylenetriamine; and
b) a pharmaceutically acceptable carrier or diluent.

68. The pharmaceutical composition of claim 67 wherein the amine monomer is represented by the following structural formula, 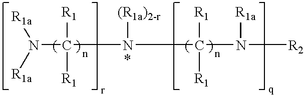
wherein:
each R₁, independently, is H or an optionally substituted alkyl group or an optionally substituted aryl group, or forms together with an R₁ bonded to an adjacent carbon or nitrogen atom and their intervening atoms an optionally substituted alicyclic, aromatic, or heterocyclic group;
each R₁ₐ is independently R₁ or 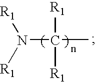
R₂ is R₁ₐ or a group represented by the following structural formula: 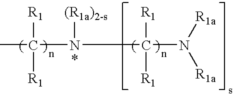
each nitrogen atom designated with “*” is optionally quarternized with R^{1a};
q is 0 or an integer from 1 to 10; r and s are 0, 1, or 2 with the proviso that the sum of r, s and q is greater than 1; and
each n, independently, is an integer from 2 to 10 with the proviso that at least one n is 2.

69. The pharmaceutical composition of claim 68, wherein the amine monomer is, represented by the following structural formula:

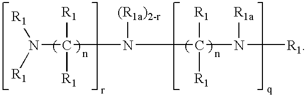

70. The pharmaceutical composition of claim 68, wherein the amine monomer is represented by the following structural formula:

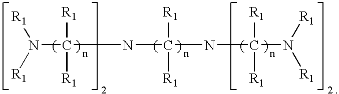

71. The pharmaceutical composition of claim 70, wherein the amine monomer is represented by the following structural formula:

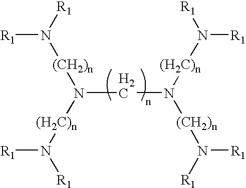

72. The pharmaceutical composition of claim 67 wherein the amine monomer is represented by the following structural formula: 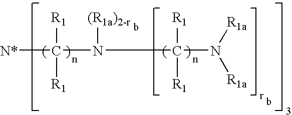
wherein each R¹, independently, is H or an optionally substituted alkyl group or an optionally substituted aryl group, or forms together with an R₁ bonded to an adjacent carbon or nitrogen atom and their intervening atoms an optionally substituted alicyclic, aromatic, or heterocyclic group;
each R^{1a} is independently R₁ or 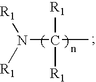
each nitrogen atom designated with “*” is optionally quarternized with R₁ₐ;
each r_{b}, independently, is 0, 1, or 2; and
each n, independently, is an integer from 2 to 10 with the proviso that at least one n is 2.

73. The pharmaceutical composition of claim 72, wherein the amine monomer is represented by the following structural formula:

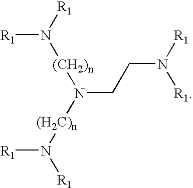

74. The pharmaceutical composition of claim 67, wherein the amine monomer is represented by the following structural formula: 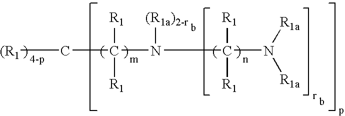
wherein each R¹, independently, is H or an optionally substituted alkyl group or an optionally substituted aryl group, or forms together with an R₁ bonded to an adjacent carbon or nitrogen atom and their intervening atoms an optionally substituted alicyclic, aromatic, or heterocyclic group;
each R₁ₐ is independently R₁ or 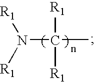
p is 1, 2, 3, or 4; each r_{b}, independently, is 0, 1, or 2 with the proviso that r_{b} is 1 or 2 if p is equal to 1;
each m, independently, is 0 or an integer from 1 to 10; and
each n, independently, is an integer from 2 to 10 with the proviso that at least one n is 2.

75. The pharmaceutical composition of claim 74 wherein the amine monomer is represented by the following structural formula:

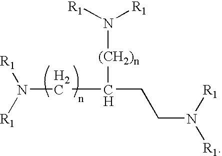

76. A method of removing phosphate from a subject comprising administering an effective amount of a pharmaceutical composition comprising:
a) a polymer or physiologically acceptable salt thereof comprising a polymerized multifunctional amine monomer wherein the amine monomer comprises at least two amine groups and at least two acyclic nitrogen atoms that are connected through a —CH₂CH₂— group, provided that the amine monomer is not ethylenediamine or diethylenetriamine; and
b) a pharmaceutically acceptable carrier or diluent.

77. The method of claim 76 wherein the amine monomer is represented by the following structural formula: 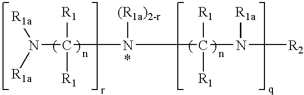
wherein:
each R₁, independently, is H or an optionally substituted alkyl group or an optionally substituted aryl group, or forms together with an R₁ bonded to an adjacent carbon or nitrogen atom and their intervening atoms an optionally substituted alicyclic, aromatic, or heterocyclic group;
each R₁ₐ is independently R₁ or 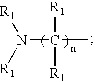
R₂ is R₁ₐ or a group represented by the following structural formula: 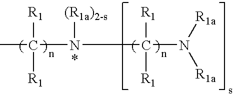
each nitrogen atom designated with “*” is optionally quarternized with R^{1a};
q is 0 or an integer from 1 to 10; r and s are 0, 1, or 2 with the proviso that the sum of r, s and q is greater than 1; and
each n, independently, is an integer from 2 to 10 with the proviso that at least one n is 2.

78. The method of claim 77, wherein the amine monomer is represented by the following structural formula:

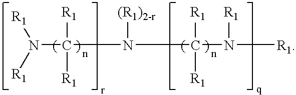

79. The method of claim 77, wherein the amine monomer is represented by the following structural formula:

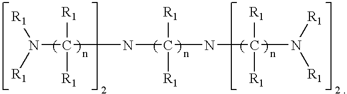

80. The method of claim 79, wherein the amine monomer is represented by the following structural formula:

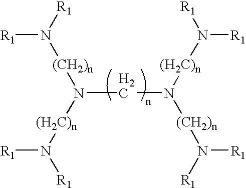

81. The method of claim 76 wherein the amine monomer is represented by the following structural formula: 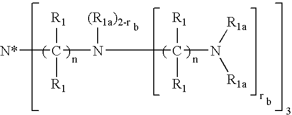
wherein each R₁, independently, is H or an optionally substituted alkyl group or an optionally substituted aryl group, or forms together with an R₁ bonded to an adjacent carbon or nitrogen atom and their intervening atoms an optionally substituted alicyclic, aromatic, or heterocyclic group;
each R₁ₐ is independently R₁ or 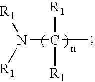
each nitrogen atom designated with “*” is optionally quarternized with R₁ₐ;
each r_{b}, independently, is 0, 1, or 2; and
each n, independently, is an integer from 2 to 10 with the proviso that at least one n is 2.

82. The method of claim 81, wherein the amine monomer is represented by the following structural formula:

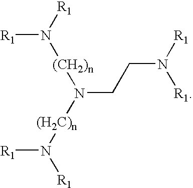

83. The method of claim 76, wherein the amine monomer is represented by the following structural formula: 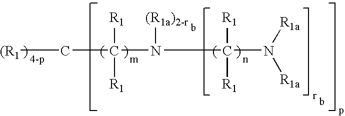
wherein each R₁, independently, is H or an optionally substituted alkyl group or an optionally substituted aryl group, or forms together with an R₁ bonded to an adjacent carbon or nitrogen atom and their intervening atoms an optionally substituted alicyclic, aromatic, or heterocyclic group;
each R₁ₐ is independently R₁ or 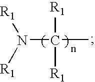
p is 1, 2, 3, or 4; each r_{b}, independently, is 0, 1, or 2 with the proviso that r_{b} is 1 or 2 if p is equal to 1;
each m, independently, is 0 or an integer from 1 to 10; and
each n, independently, is an integer from 2 to 10 with the proviso that at least one n is 2.

84. The method of claim 83 wherein the amine monomer is represented by the following structural formula: [chemical structure]
